(19) United States
(12) Patent Application Publication
(10) Pub. No.: US 2025/0069742 A1
(43) Pub. Date: Feb. 27, 2025

(54) REMOTE AREA AUTOMATIC TIME CORRECTION METHOD

(71) Applicant: FAR EAST MEDICAL ELECTRONICS TECHNOLOGY CO., LTD., New Taipei (TW)

(72) Inventors: CHUN-NAN TENG, New Taipei (TW); JUI-HAN CHANG, New Taipei (TW)

(21) Appl. No.: 18/784,166

(22) Filed: Jul. 25, 2024

(30) Foreign Application Priority Data
Aug. 21, 2023 (TW) 112131375

(51) Int. Cl. G16H 40/67; A61B 5/00; G16H 20/10

(52) U.S. Cl. CPC G16H 40/67; A61B 5/0002; G16H 20/10

(57) ABSTRACT

A remote area automatic time correction method, in which the user at the remote end performs physiological measurement through an inspection instrument. The obtained physiological measurement value information is transmitted to a wireless transceiver unit of an automatic correction system through a wireless transmission unit for a control unit of the automatic correction system to detect the longitude and latitude location of the area of the remote user, so as to obtain the time difference between the time in the area of the remote user and the location of the automatic correction system. The control unit converts the time that the user should perform the treatment, and then converts it into the time in the area of the user is located. When the time for the treatment is reached, a warning unit of the remote end reminds the user to proceed with the treatment.

This application claims the priority benefit of Taiwan patent application number 112131375 filed on Aug. 21, 2023.

### BACKGROUND OF THE INVENTION

### 1. Field of the Invention

The present invention provides a remote area automatic time correction method, especially an automatic correction method for longitude and latitude of a non-land remote location and converting time differences in different areas. The time difference is converted through the longitude and latitude of the remote area to achieve the purpose of effectively controlling the user's treatment course.

### 2. Description of the Related Art

People inevitably suffer from illnesses in their daily lives, and they usually go directly to various hospitals or clinics to seek help from medical staff for diagnosis and treatment. Depending on the various physical ailments, you can take medicines, inject injections or apply medicines to relieve the physical ailments and obtain healing. It is very simple and convenient for medical diagnosis and treatment.

However, cargo ships, cruise ships or ocean-going fishing boats and various ships sailing on the ocean that are located on the ocean for long periods of time, in response to various work or travel needs, staff on various ships and vessels will be away from land for long periods of time. Living on various ships and vessels on the ocean brings many inconveniences in daily life, and once pain occurs, immediate and effective treatment cannot be obtained. Due to factors such as funding, most ships are not equipped with medical-related personnel. Once someone on the ship becomes ill, it is impossible to immediately provide medical treatment and medication to the patient. If the patients on the ship are mildly ill, they may be able to endure the pain until the ship docks before seeking medical treatment. However, those with more serious conditions sometimes have to be transported ashore by helicopter for treatment, which often causes the inconvenience of ships sailing on the sea for long periods of time.

In order to solve the problem for people who do not live on land for long periods of time, if they have medical needs due to various illnesses, they can connect with medical institutions through video consultation. Then, the doctor can conduct inquiry and diagnosis through video consultation, and the doctor will issue a prescription based on the video diagnosis and treatment content, so that the user can obtain the medical drugs provided on the ship and treat the disease. However, the taking or use of medical drugs must be limited to a certain time in order to achieve the desired effect, for example, predetermine a certain number of hours at which medication must be taken, how long before or after a meal when medication must be taken, etc. However, ships that are on the ocean for long periods of time usually cross different time zones due to constant movement, resulting in different time difference changes. The staff on the ship will be confused about the time when they must take the medicine. It is easy to misjudge the time due to different time differences, resulting in the time of taking the medicine too fast or too slow, which will affect the treatment of the disease.

Therefore, how to solve the problem of not taking medicine immediately for people who are currently traveling on ships on the sea for long periods of time due to the time difference in the location, which also causes the inconvenience and shortcomings of the users who cannot be treated as soon as possible for their illnesses on the ship is the direction that relevant manufacturers in this industry are eager to study and improve.

### SUMMARY OF THE INVENTION

In view of the above-mentioned problems and deficiencies, the inventor collected relevant information, and after various evaluations and considerations, he designed this remote area automatic time correction method.

The main object of the present invention is to provide a remote area automatic time correction method in which the user of the remote end performs physiological measurements through an inspection instrument. The physiological measurement value information obtained by the user through the inspection instrument can be transmitted to the wireless transceiver unit of the automatic correction system through the wireless transmission unit, so that the control unit of the automatic correction system can use the physiological measurement value information received from the wireless transmission unit to detect the longitude and latitude location of the area where the remote end is located, so as to obtain the time difference between the time in the area where the remote end is located and the location of the automatic correction system. The control unit of the automatic correction system then uses the medical-related information of the user in the storage unit to calculate the time that the user should undergo treatment, and then converts it into the time in the area where the user is located. When it is time for the user to perform the treatment, the automatic correction system will remind the user to perform the treatment through the warning unit of the remote end to avoid delays in medication and treatment time, so as to achieve the purposes of monitoring and managing medical-related treatment time for the user at the remote end.

Another object of the present invention is that in the remote area automatic time correction method, the remote end can be a ship, a cargo ship, an ocean-going fishing vessel or a cruise ship located in the ocean; the inspection instrument can be a blood pressure machine, an oximeter, a body temperature machine or a blood glucose meter, the physiological measurement can be carried out to measure and examine the blood pressure, pulse, heartbeat, body temperature, blood oxygen or blood sugar of the physiological status of the user, and the physiological measurement value information can be the numerical information of the blood pressure, pulse, heartbeat, body temperature, blood oxygen or blood sugar and other physiological status measurement and examination information of the user.

Still another object of the present invention is that in the remote area automatic time correction method, the control unit of the automatic correction system detects the longitude and latitude location of the ocean position in the area where the remote end is located and further identifies the time zone and azimuth of the area where the remote end is located, and the location of automatic correction system can be the land area, so that the time difference between the standard time of the longitude and latitude of the location where the remote end is located and the standard time of the location of the automatic correction system can be calculated.

Still another object of the present invention is that in the remote area automatic time correction method, the medical-related treatment time can be the medical-related treatment time when the user should take medicines (oral medicines), injections, eye drops, etc., or use external medicines (applying or changing dressings and bandaging on external injuries).

### BRIEF DESCRIPTION OF THE DRAWINGS

FIG. 1 is a system block diagram of the present invention.

FIG. 2 is a flow chart of the present invention.

### DETAILED DESCRIPTION OF THE PREFERRED EMBODIMENT

In order to achieve the above-mentioned objectives and effects, the technical means and implementation method adopted by the present invention are described in detail below in order to facilitate a complete understanding.

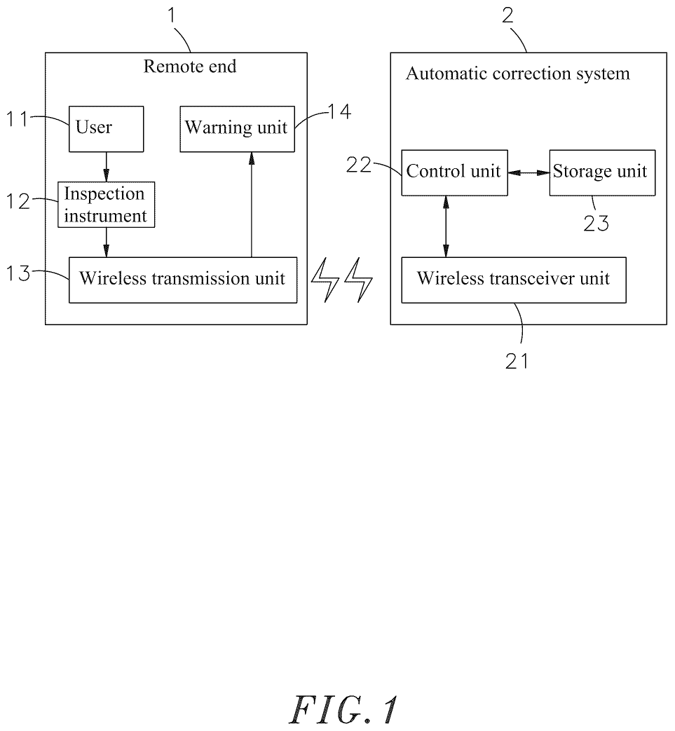

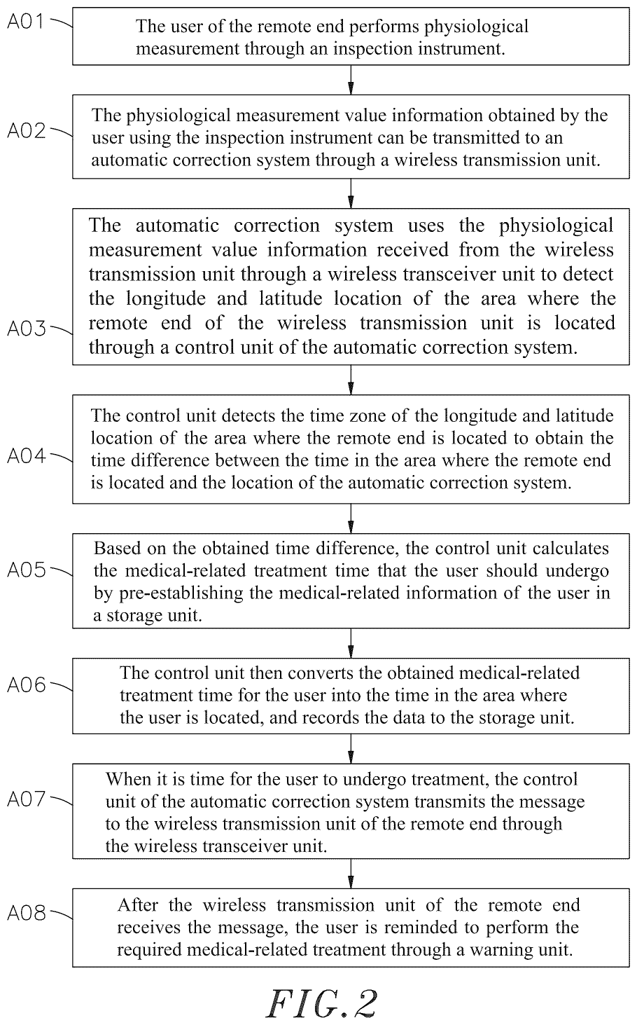

Please refer to FIGS. 1 and 2, which are system block diagram of the present invention and flow chart of the present invention. It can be clearly seen from the figures that the remote area automatic time correction method of the present invention can be implemented according to the following steps:

(A01) The user 11 of the remote end 1 performs physiological measurement through an inspection instrument 12.

(A02) Then the physiological measurement value information obtained by the user 11 using the inspection instrument 12 can be transmitted to an automatic correction system 2 through a wireless transmission unit 13.

(A03) The automatic correction system 2 uses the physiological measurement value information received from the wireless transmission unit 13 through a wireless transceiver unit 21 to detect the longitude and latitude location of the area where the remote end 1 of the wireless transmission unit 13 is located through a control unit 22 of the automatic correction system 2.

(A04) The control unit 22 detects the time zone of the longitude and latitude location of the area where the remote end 1 is located to obtain the time difference between the time in the area where the remote end 1 is located and the location of the automatic correction system 2.

(A05) Based on the obtained time difference, the control unit 22 calculates the medical-related treatment time that the user 11 should undergo by pre-establishing the medical-related information of the user 11 in a storage unit 23.

(A06) The control unit 22 then converts the obtained medical-related treatment time for the user 11 into the time in the area where the user 11 is located, and records the data to the storage unit 23.

(A07) When it is time for the user 11 to undergo treatment, the control unit 22 of the automatic correction system 2 transmits the message to the wireless transmission unit 13 of the remote end 1 through the wireless transceiver unit 21.

(A08) After the wireless transmission unit 13 of the remote end 1 receives the message, the user 11 is reminded to perform the required medical-related treatment through a warning unit 14.

The above-mentioned remote end 1 of the step (A01) of the present invention can be a ship, cargo ship, ocean-going fishing vessel or cruise ship located in the ocean, and the user 11 can be a crew member, staff or tourist located at the remote end 1. The inspection instrument 12 in the steps (A01) and (A02) can be a blood pressure machine, an oximeter, a body temperature machine or a blood glucose meter, etc., and the physiological measurement can be carried out to measure and examine the physiological status of the user 11 such as blood pressure, pulse, heartbeat, body temperature, blood oxygen or blood sugar, etc. The physiological measurement value information may be numerical information of blood pressure, pulse, heartbeat, body temperature, blood oxygen or blood sugar and other physiological status measurement and examination information of the user 11.

The above-mentioned control unit 22 of the automatic correction system 2 of the present invention detects the longitude and latitude location of the ocean position on the earth in the area where the remote end 1 is located. The control unit 22 further identifies the time zone and azimuth of the area where the remote end 1 is located, and the location of automatic correction system 2 can be the land area on the earth, so that the time difference between the standard time of the longitude and latitude of the location where the remote end 1 is located (such as the Atlantic, Pacific or Indian Ocean, etc.) on the earth and the standard time of the location of the automatic correction system 2 can be calculated. Through the automatic correction system 2 for the purposes of automatic correction and verification, etc., the user 11 can be provided with correct time information.

Furthermore, between the above-mentioned wireless transceiver unit 21 and the wireless transmission unit 13 in the steps (A02) to (A08) of the present invention, the wireless signal transmission can be carried out through the Global Positioning System (GPS), so that the numerical information such as blood pressure, pulse, heartbeat, body temperature, blood oxygen or blood sugar and other physiological status measurement and examination information of the user 11 at the remote end 1 can be transmitted to the wireless transceiver unit 21 of the automatic correction system 2 through the wireless transmission unit 13, and can be stored in storage unit 23. This can achieve the management and monitoring of the physiological status of the user 11, and can also be used as a reference for medical staff to check the physiological health status of the user 11. The control unit 22 of the steps (A03) to (A07) can be a central processing unit (CPU), a microprocessor or a chip.

In addition, the medical-related treatment time of the above-mentioned steps (A05) to (A07) of the present invention can be the medical-related treatment time when the user 11 should take medicines (oral medicines), injections, eye drops, etc., or use external medicines (applying or changing dressings and bandaging on external injuries).

In addition, the above-mentioned storage unit 23 of the automatic correction system 2 in the step (A05) of the present invention can be used to pre-establish medical-related information of each user 11 at the remote end 1 such as records of chronic diseases, scheduled diagnosis and treatment time or injury records, so as to control the medical-related information of each user 11 at the remote end 1. The automatic correction system 2 can remind the user 11 to perform medical-related treatment courses through the warning unit 14 of the remote end 1 to avoid delays in medication, treatment schedule, etc., so as to achieve the purposes of monitoring and managing the medical-related treatment time of the user 11 at the remote end 1.

The above-mentioned warning unit 14 of the step (A08) of the present invention can be a smart watch, a smart phone, a tablet computer or a radio walkie-talkie, etc., so that the user 11 can be reminded of the required medical-related treatment time through warning sounds, vibrations or voice prompts.

The above is only a preferred embodiment of the present invention, and does not limit the patent scope of the present invention. Therefore, all simple modifications and equivalent structural changes made by using the contents of the description and drawings of the present invention shall be included in the patent scope of the present invention and shall be clearly stated.

To sum up, the remote area automatic time correction method of the present invention can really achieve its effect and purpose when used. Therefore, the present invention is sincerely an invention with excellent practicability. In order to meet the requirements for application for a new patent, I would like to file an application in accordance with the law. I hope that the review committee will approve this case as soon as possible to protect the hard work of the inventor. If the review committee has any doubts, please don't hesitate to send us a letter to instruct, the inventor will try his best to cooperate, thank you for your help.

What the invention claimed is:

1. A remote area automatic time correction method, comprising the steps of:
(A01) an user of a remote end performing physiological measurement through an inspection instrument;
(A02) transmitting the physiological measurement value information obtained by said user using said inspection instrument to an automatic correction system through a wireless transmission unit;
(A03) said automatic correction system using the physiological measurement value information received from said wireless transmission unit through a wireless transceiver unit to detect the longitude and latitude location of the area where said remote end of said wireless transmission unit is located through a control unit of said automatic correction system;
(A04) said control unit detecting the time zone of the longitude and latitude location of the area where said remote end is located to obtain the time difference between the time in the area where said remote end is located and the location of said automatic correction system;
(A05) based on the obtained time difference, said control unit calculating the medical-related treatment time that said user needs to undergo by pre-establishing the medical-related information of said user in a storage unit;
(A06) said control unit then converting the obtained medical-related treatment time for said user into the time in the area where said user is located, and recording the data to said storage unit;
(A07) said control unit of said automatic correction system transmitting the message to said wireless transmission unit of said remote end through said wireless transceiver unit when the time said user needs to undergo the treatment is reached; and
(A08) said wireless transmission unit using a warning unit to remind said user to perform the medical-related treatment.

2. The remote area automatic time correction method as claimed in claimed 1, wherein said remote end in the step (A01) is a ship, cargo ship, ocean-going fishing vessel or cruise ship located in the ocean.

3. The remote area automatic time correction method as claimed in claimed 1, wherein said inspection instrument in the steps (A01) and (A02) is selectively a blood pressure machine, an oximeter, a body temperature machine or a blood glucose meter, and the physiological measurement is carried out to measure and examine the blood pressure, pulse, heartbeat, body temperature, blood oxygen or blood sugar of the physiological status of said user.

4. The remote area automatic time correction method as claimed in claimed 1, wherein the numerical information of the physiological measurement value information in the steps (A02) and (A03) is the numerical information of the physiological status measurement value information of the blood pressure, pulse, heartbeat, body temperature, blood oxygen or blood sugar of said user.

5. The remote area automatic time correction method as claimed in claimed 1, wherein the wireless signal transmission is carried out between said wireless transceiver unit and said wireless transmission unit in the step (A03) through the Global Positioning System (GPS).

6. The remote area automatic time correction method as claimed in claimed 1, wherein said control unit in the steps (A03) to (A07) is selectively a central processing unit (CPU), a microprocessor or a chip.

7. The remote area automatic time correction method as claimed in claimed 1, wherein said control unit in the step (A04) detects the longitude and latitude location of the ocean position in the area where said remote end is located, and the location of said automatic correction system is a land area, so that the time difference between the standard time of the longitude and latitude of the location in the area where said remote end is located and the standard time of the location of said automatic correction system is calculated.

8. The remote area automatic time correction method as claimed in claimed 1, wherein the medical-related treatment time in the steps (A05) to (A07) is when said user needs to take medicines (oral medicines), injections, eye drops, etc., or use external medicines (applying or changing dressings and bandaging on external injuries).

9. The remote area automatic time correction method as claimed in claimed 1, wherein said warning unit in the step (A08) is selectively a smart watch, a smart phone, a tablet computer or a radio walkie-talkie.
